(19) United States
(12) Patent Application Publication
(10) Pub. No.: US 2021/0363482 A1
(43) Pub. Date: Nov. 25, 2021

(54) THREE-DIMENSIONAL CULTURE METHOD, THREE-DIMENSIONAL CULTURE STRUCTURE, AND THREE-DIMENSIONAL CULTURE STRUCTURE MANUFACTURING METHOD

(71) Applicants: Sharp Kabushiki Kaisha, Sakai City, Osaka (JP); TOTTORI INSTITUTE OF INDUSTRIAL TECHNOLOGY, Tottori-shi, Tottori (JP)

(72) Inventors: Tokio TAGUCHI, Sakai City (JP); Yasuhiro SHIBAI, Sakai City (JP); Mitsuaki SUGINE, Yonago-shi (JP); Isao TAKEBAYASHI, Yonago-shi (JP); Yuko SUGIMOTO, Sakaiminato-shi (JP)

(21) Appl. No.: 17/051,461

(22) PCT Filed: Aug. 9, 2019
(86) PCT No.: PCT/JP2019/031657

§ 371 Date: Oct. 29, 2020

(30) Foreign Application Priority Data
Sep. 21, 2018 (JP) 2018-177227

(51) Int. Cl. C12N 5/00

(52) U.S. Cl. CPC C12N 5/0062; C12N 5/0068; C12N 2533/30; C12N 2525/00; C12N 2513/00

(57) ABSTRACT

A three-dimensional culture method includes: providing a cell suspension, the cell suspension containing a cell (12C) and a culture medium (14M); providing a solid surface (10S), the solid surface having a plurality of raised portions (10Sp) whose height is not less than 10 nm and not more than 1 mm; attaching a liquid drop (16D) of the cell suspension to the solid surface (10S); and culturing the cell (12C) in the liquid drop (16D) under such conditions that a direction of gravity exerted on the liquid drop (16D) is toward the solid surface (10S).

### TECHNICAL FIELD

The present invention relates to a three-dimensional cell culture method (hereinafter, referred to as “three-dimensional culture method”), a structure for use in three-dimensional culture (including container), and a method for producing a structure for three-dimensional culture.

### BACKGROUND ART

In recent years, three-dimensional cell culture methods (hereinafter, “three-dimensional culture methods”) have been receiving attention as a technique indispensable for drug discovery and regenerative medicine (for example, Patent Documents No. 1 to No. 4, Non-patent Documents No. 1 to No. 3).

The three-dimensional culture method is a method for culturing cells in vitro in such a manner that the cells interact three-dimensionally with each other, resulting in a spheroid in which the properties of the cells in vivo are reflected well. The spheroid generated by the three-dimensional culture method can express, in a manner closer to a living organism, the properties and functions of the biological tissue from which cultured cells were originated, than cells generated by a two-dimensional culture method. In this specification, such a characteristic of the spheroid is referred to as “reproducibility of tissue”. When a protein generated by intracellular gene expression physiologically functions in a manner closer to a living organism, it is mentioned as higher reproducibility of tissue.

As the three-dimensional culture method, culture methods with the use of a surface which has a minute uneven structure are disclosed in, for example, Patent Documents No. 1 to No. 3 and Non-patent Document No. 1. The culture methods disclosed in these documents are such that a cell suspension which contains cells and a culture medium (herein, referring to liquid culture medium) is poured into a container which has a minute uneven structure at the bottom surface, and culturing is carried out while some of the cells are adhered to the bottom surface of the container in the liquid. Hereinafter, in this specification, the culture methods disclosed in Patent Documents No. 1 to No. 3 and Non-patent Document No. 1 are referred to as “low-adhesive three-dimensional culture method”.

Hanging drop methods by which cells are cultured in a liquid drop are disclosed in, for example, Patent Document No. 4 and Non-patent Documents No. 2 and No. 3.

### CITATION LIST

### Patent Literature

Patent Document No. 1: Japanese Laid-Open Patent Publication No. 2005-168494
Patent Document No. 2: WO 2007/097120
Patent Document No. 3: WO 2017/126589
Patent Document No. 4: WO 2007/114351
Patent Document No. 5: Japanese Patent No. 4265729
Patent Document No. 6: Japanese Laid-Open Patent Publication No. 2009-166502
Patent Document No. 7: WO 2011/125486
Patent Document No. 8: WO 2013/183576
Patent Document No. 9: WO 2015/163018

### Non-Patent Literature

Non-patent Document No. 1: Yoshii Y, Furukawa T, Aoyama H, Adachi N, Zhang M R, Wakizaka H, Fujibayashi Y, Saga T, “Regorafenib as a potential adjuvantchemotherapy agent in disseminated small colon cancer: Drug selection outcome of anovel screening system using nanoimprinting 3-dimensional culture with HCT116-RFP cells”, Int. J. Oncol., 2016 April; 48(4):1477-84.
Non-patent Document No. 2: Singla D K and Sobel B E, Biochem Biophys Res Commun. 2005 335(3):637-42
Non-patent Document No. 3: Foty, R., “A Simple Hanging Drop Cell Culture Protocol for Generation of 3D Spheroids”. JoVE., 51, 2720 (2011).

### SUMMARY OF INVENTION

### Technical Problem

However, according to research by the present inventors, the above-described conventional three-dimensional culture methods still have room for improvement in operational easiness or mass productivity. In addition, development of a three-dimensional culture method which is capable of generating a spheroid with further-improved reproducibility of tissue has been demanded.

In the low-adhesive three-dimensional culture method with the use of the minute uneven structure at the bottom surface, the minute uneven structure at the bottom surface serves as a scaffold for cells. When the interaction between the uneven structure at the bottom surface and the cells is stronger than the interaction between the cells, the cells cannot sufficiently proliferate in the thickness direction, and proliferation in the in-plane direction is dominant. As a result, in some cases, a three-dimensional tissue structure cannot be sufficiently reproduced. Further, in the low-adhesive three-dimensional culture method, cells repeatedly contact and adhere with each other while the cells randomly migrate in the in-plane direction, and generate a spheroid with the occurrence of cytokinesis. Therefore, disadvantageously, the number of cells included in the spheroid largely varies, and the reproducibility in shape and size of the spheroid is low.

According to the hanging drop method, culture is carried out in a liquid drop and therefore the number of cells can be easily controlled, and advantageously, the reproducibility in shape and size of the spheroid is high. However, there is no surface in the liquid drop that can serve as a scaffold for cells and, therefore, cell types which are highly dependent on scaffold sometimes cannot maintain viability. In the hanging drop method, a surface with the liquid drop attached thereto faces down (faces in the gravity direction) and, therefore, the operational easiness is disadvantageously low.

Although the reproducibility of tissue of a spheroid generated by any of the above-described three-dimensional culture methods is higher than the reproducibility of tissue of a spheroid generated by a two-dimensional culture method (two-dimensional culture method), further improvement has been demanded.

In view of the foregoing, an object of an embodiment of the present invention is to provide a three-dimensional culture method which is better in operational easiness or mass productivity and/or which is capable of generating a spheroid with higher reproducibility of tissue than the conventional three-dimensional culture methods. An object of another embodiment of the present invention is to provide a structure for three-dimensional culture and/or a method for producing a structure for three-dimensional culture, which are suitably used in such a three-dimensional culture method.

### Solution to Problem

According to an embodiment of the present invention, solutions as described in the following Items are provided.

[Item 1]

A three-dimensional culture method including: providing a cell suspension, the cell suspension containing a cell and a culture medium; providing a solid surface, the solid surface having a plurality of raised portions whose height is not less than 10 nm and not more than 1 mm; attaching a liquid drop of the cell suspension to the solid surface; and culturing the cell in the liquid drop under such conditions that a direction of gravity exerted on the liquid drop is toward the solid surface.

[Item 2]

The method of Item 1 wherein, when viewed in a normal direction of the solid surface, a two-dimensional size of the plurality of raised portions is in the range of not less than 10 nm and not more than 500 nm.

[Item 3]

The method of Item 1 or 2, wherein the height of the plurality of raised portions is not less than 10 nm and not more than 500 nm.

[Item 4]

The method of any of Items 1 to 3, wherein an adjoining distance of the plurality of raised portions is not less than 10 nm and not more than 1000 nm. The adjoining distance of the plurality of raised portions may be not more than 500 nm.

[Item 5]

The method of any of Items 1 to 4, wherein the plurality of raised portions have a generally-conical tip portion.

[Item 6]

The method of any of Items 1 to 5, wherein a contact angle of the solid surface with respect to the cell suspension is not less than 17°. It may be at least required that, after the lapse of 10 seconds since placement of the drop, the contact angle of the solid surface with respect to the cell suspension is not less than 17°.

[Item 7]

The method of any of Items 1 to 6, wherein a contact angle of the solid surface with respect to the cell suspension is not less than 90°. It may be at least required that, after the lapse of 10 seconds since placement of the drop, the contact angle of the solid surface with respect to the cell suspension is not less than 90°.

[Item 8]

The method of any of Items 1 to 7, wherein a sliding angle of the solid surface with respect to the cell suspension is not less than 45°. The sliding angle may be evaluated based on a value after the lapse of 20 seconds since placement of the drop.

[Item 9]

The method of any of Items 1 to 8, wherein the solid surface is made of a synthetic polymer.

[Item 10]

The method of any of Items 1 to 9, wherein a volume of the liquid drop is not less than 10 μL and not more than 50 μL.

From the viewpoint of formation of a liquid drop in an appropriate shape and manageability, the above-described ranges are preferred.

[Item 11]

The method of any of Items 1 to 10, wherein a seeding density of the cell contained in the liquid drop is not less than 10³ cells/mL and not more than 10⁷ cells/mL.

[Item 12]

The method of any of Items 1 to 11, wherein a height of the liquid drop is not less than 1 mm.

[Item 13]

The method of any of Items 1 to 12, further including adding the culture medium into the liquid drop while the cell is cultured in the liquid drop.

[Item 14]

The method of Item 13 further including, before adding the culture medium, aspirating part of the culture medium from the liquid drop.

According to other embodiments of the present invention, solutions as described in the following Items are provided.

[Item 15]

A structure for three-dimensional culture having a solid surface for use in the three-dimensional culture method as set forth in any of Items 1 to 14.

The structure for three-dimensional culture is provided as a part of a container.

[Item 16]

A method for producing a structure for three-dimensional culture, the structure for three-dimensional culture having the solid surface, the structure for three-dimensional culture including on the solid surface a spheroid cultured using the three-dimensional culture method as set forth in any of Items 1 to 14.

A spheroid cultured using the three-dimensional culture method as set forth in any of Items 1 to 14 can be provided together with the structure for three-dimensional culture (e.g., container).

### Advantageous Effects of Invention

According to an embodiment of the present invention, a three-dimensional culture method is provided which is better in operational easiness or mass productivity and/or which is capable of generating a spheroid with higher reproducibility of tissue than the conventional three-dimensional culture methods. According to another embodiment of the present invention, a structure for three-dimensional culture which is suitably used in such a three-dimensional culture method is provided. According to still another embodiment of the present invention, a structure for three-dimensional culture (e.g., container) is provided which has at a surface a spheroid with higher reproducibility of tissue than conventional ones.

### BRIEF DESCRIPTION OF DRAWINGS

FIG. 1 is a diagram schematically showing a state of culture in a three-dimensional culture method of an embodiment of the present invention.

FIG. 2A is a schematic cross-sectional view of a synthetic polymer film 34A which has at a surface a moth-eye structure for use in the three-dimensional culture method of an embodiment of the present invention.

FIG. 2B is a schematic cross-sectional view of a synthetic polymer film 34B which has at a surface a moth-eye structure for use in the three-dimensional culture method of an embodiment of the present invention.

FIG. 3A shows an optical microscopic image of a result of drop culturing (left) and an optical microscopic image of a result of two-dimensional culturing (right) of human liver cancer cell line HepG2.

FIG. 3B shows a top image (left) and a side image (right) by an electron microscope of a HepG2 spheroid generated by a drop culture method.

FIG. 3C is a graph showing the number of viable cells of liver cancer cell line HepG2 during drop culturing.

FIG. 3D is a graph showing the evaluated CYP activity of a liver cancer cell HepG2 spheroid generated by the drop culture method.

FIG. 4A shows an optical microscopic image of a result of drop culturing (left) and an optical microscopic image of a result of two-dimensional culturing (right) of human embryonic kidney cells HEK293.

FIG. 4B shows an optical microscopic image of a result of drop culturing (left) and an optical microscopic image of a result of two-dimensional culturing (right) of mouse embryonic fibroblast 3T3-L1.

FIG. 4C shows an optical microscopic image of a result of drop culturing (left) and an optical microscopic image of a result of two-dimensional culturing (right) of mouse mesenchymal stem cells C3H10t1/2.

FIG. 4D shows an optical microscopic image of a result of drop culturing (left) and an optical microscopic image of a result of two-dimensional culturing (right) of mouse myoblast cells C2C12.

FIG. 5A shows an optical microscopic image of spheroids (Level 1) generated by a drop culture method.

FIG. 5B shows optical microscopic images of spheroids (Level 2) generated by a drop culture method.

FIG. 5C shows optical microscopic images of spheroids (Level 3) generated by a drop culture method.

FIG. 5D shows an optical microscopic image of spheroids (Level 4) generated by a drop culture method.

FIG. 5E shows optical microscopic images of spheroids (Level 5) generated by a drop culture method.

### DESCRIPTION OF EMBODIMENTS

Hereinafter, a three-dimensional culture method, a structure for three-dimensional culture and a method for producing a structure for three-dimensional culture of an embodiment of the present invention are described.

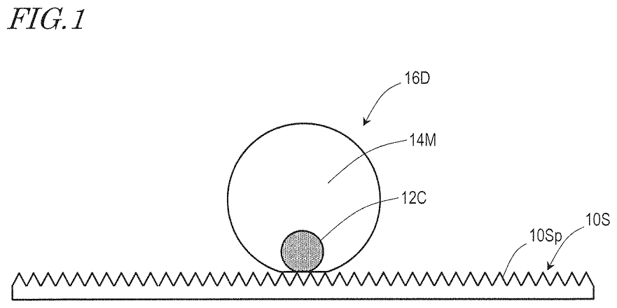

The three-dimensional culture method of an embodiment of the present invention includes, as schematically shown in FIG. 1, attaching a liquid drop 16D of a cell suspension which contains a cell 12C and a culture medium 14M to a solid surface 10S and culturing the cell 12C in the liquid drop 16D under such conditions that the direction of the gravity exerted on the liquid drop 16D is toward the solid surface 105. In this three-dimensional culture method (hereinafter, referred to as “drop culture method”), the cell 12C is cultured in the liquid drop 16D and, therefore, the number of cells can be easily controlled, and the reproducibility in shape and size of the spheroid is high, while the hanging drop method also have these advantages. Since the cell 12C is cultured under such conditions that the direction of the gravity exerted on the liquid drop 16D is toward the solid surface 10S, the solid surface 10S serves as a scaffold and, therefore, even cell types of high scaffold dependency can maintain relatively-high viability. Since the surface 10S with the liquid drop 16D attached thereto is not necessary to face down (the surface 10S is not necessary to face in the gravity direction), operational easiness is high as compared with the hanging drop method. The solid surface 10S has a plurality of raised portions 10Sp which can serve as a scaffold.

The liquid drop 16D, exclusive of a portion which is in contact with the solid surface 10S, is in contact with an atmosphere gas (e.g., air) and forms a closed culture space. In FIG. 1, the bottom surface of the liquid drop 16D is shown to be in contact with the tips of the raised portions 10Sp and the liquid drop 16D is shown to be present only above the tips of the raised portions 10Sp, although part of the bottom portion of the liquid drop 16D may be present in a gap between adjoining raised portions 10Sp. The volume of the liquid drop 16D is, for example, not less than 10 μL and not more than 50 μL.

The solid surface 10S which enables formation of a stable liquid drop 16D and efficient culturing of cells is, for example, a solid surface which has a plurality of raised portions 10Sp whose height is not less than 10 nm and not more than 1 mm, as confirmed by the experimental results shown later. As also disclosed in, for example, Patent Document No. (registered as Japanese Patent No. 4507845), using a solid surface which has a plurality of raised portions whose height is not less than 10 nm and not more than 1 mm enables three-dimensional culture. However, as previously described, in the low-adhesive three-dimensional culture method which utilizes the minute uneven structure at the bottom surface, the three-dimensional tissue structure cannot be sufficiently reproduced, or the reproducibility in shape and size of the spheroid is low.

According to the drop culture method, cells in the liquid drop 16D are cultured. Therefore, the drop culture method is free from the above-described disadvantages of the low-adhesive three-dimensional culture method. The cells 12C aggregate in the three-dimensionally closed liquid drop 16D under the influence of the gravity by accumulating on the bottom surface which is in contact with the solid surface 10S. Therefore, a certain amount of cells interact with the plurality of raised portions 10Sp of the solid surface 10S and, over these cells, there are other cells interact with each other. As a result, in our estimation, unlike the low-adhesive three-dimensional culture, proliferation also occurs appropriately in the thickness direction, resulting in a spheroid in which the reproducibility of the three-dimensional tissue structure is high.

Hereinafter, the three-dimensional culture method (drop culture method) of an embodiment of the present invention is described with experimental examples where the solid surface 10S has a moth-eye structure. A solid surface with a moth-eye structure, which has been developed by one of the present applicants as an antireflection film or a microbicidal synthetic polymer film, can be suitably used in the drop culture method. The disclosures of Patent Documents No. 5 to No. 8 (antireflection film) and Patent Document No. 9 (microbicidal synthetic polymer film) are incorporated by reference in this specification.

As disclosed in Patent Documents No. 5 to No. 9, using an anodized porous alumina layer enables production of a synthetic polymer film (for example, a photocured resin film formed by curing a photocurable resin or a thermoset resin film formed by curing a thermosetting resin) which has a moth-eye structure at a surface with high mass productivity. In the experimental examples described below, a photocured resin film used has a moth-eye structure formed by the above-described method at a surface and has the features previously described in Items 2-9. Note that, however, as disclosed in Patent Document No. 1, the size and height of the plurality of raised portions and the distance between adjoining raised portions (the pitch if the raised portions are regularly arrayed) are not limited to these examples. The material of the moth-eye structure may be any of organic materials and inorganic materials.

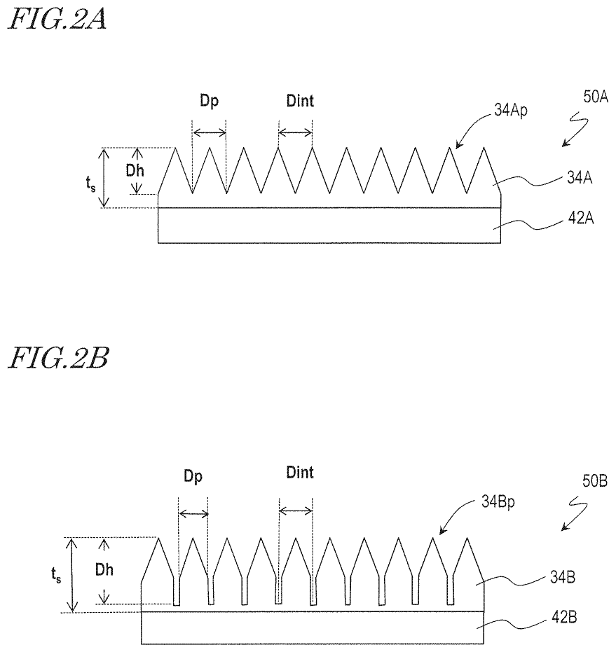

The configurations of synthetic polymer films 34A and 34B which have a moth-eye structure at a surface for use in the drop culture method are described with reference to FIG. 2A and FIG. 2B. The synthetic polymer films 34A and 34B are examples of the structure for three-dimensional culture of an embodiment of the present invention.

FIG. 2A and FIG. 2B show schematic cross-sectional views of the synthetic polymer films 34A and 34B, respectively. The synthetic polymer films 34A and 34B described herein as examples are formed on base films 42A and 42B, respectively, although the present invention is not limited to these examples. The synthetic polymer films 34A and 34B can be directly formed on a surface of an arbitrary object.

A film 50A shown in FIG. 2A includes a base film 42A and a synthetic polymer film 34A provided on the base film 42A. The synthetic polymer film 34A has a plurality of raised portions 34Ap over its surface. The plurality of raised portions 34Ap constitute a moth-eye structure. When viewed in a normal direction of the synthetic polymer film 34A, the two-dimensional size of the raised portions 34Ap, Dₚ, is in the range of not less than 10 nm and not more than 500 nm. Here, the “two-dimensional size” of the raised portions 34Ap refers to the diameter of a circle equivalent to the area of the raised portions 34Ap when viewed from the normal of the surface. When the raised portions 34Ap have a conical shape, for example, the two-dimensional size of the raised portions 34Ap is equivalent to the diameter of the base of the cone. The typical adjoining distance of the raised portions 34Ap, Dᵢₙₜ, is not less than 10 nm and not more than 1000 nm. When the raised portions 34Ap are densely arranged so that there is no gap between adjoining raised portions 34Ap (e.g., the bases of the cones partially overlap each other) as shown in FIG. 2A, the two-dimensional size of the raised portions 34Ap, Dₚ, is equal to the adjoining distance Dᵢₙₜ. The typical height of the raised portions 34Ap, Dₕ, is not less than 10 nm and not more than 500 nm. The thickness of the synthetic polymer film 34A, tₛ, is not particularly limited but only needs to be greater than the height Dₕ of the raised portions 34Ap.

The synthetic polymer film 34A shown in FIG. 2A has the same moth-eye structure as the antireflection films disclosed in Patent Documents No. 5 to No. 8. From the viewpoint of producing an antireflection function, it is preferred that the surface has no flat portion, and the raised portions 34Ap are densely arranged over the surface. Further, the raised portions 34Ap preferably has a such shape that the cross-sectional area (a cross section parallel to a plane which is orthogonal to an incoming light ray, e.g., a cross section parallel to the surface of the base film 42A) increases from the air side to the base film 42A side, e.g., a conical shape. From the viewpoint of suppressing interference of light, it is preferred that the raised portions 34Ap are arranged without regularity, preferably randomly. However, these features are unnecessary when the synthetic polymer film 34A is used for drop culturing. For example, the raised portions 34Ap do not need to be densely arranged. The raised portions 34Ap may be regularly arranged. The upper limit values and the lower limit values of Dₚ, Dᵢₙₜ and Dₕ may exceed the wavelength range of visible light because it is not necessary to prevent reflection of visible light.

A film 50B shown in FIG. 2B includes a base film 42B and a synthetic polymer film 34B provided on the base film 42B. The synthetic polymer film 34B has a plurality of raised portions 34Bp over its surface. The plurality of raised portions 34Bp constitute a moth-eye structure. In the film 50B, the configuration of the raised portions 34Bp of the synthetic polymer film 34B is different from that of the raised portions 34Ap of the synthetic polymer film 34A of the film 50A. Descriptions of features which are common with those of the film 50A are sometimes omitted.

When viewed in a normal direction of the synthetic polymer film 34B, the two-dimensional size of the raised portions 34Bp, Dₚ, is in the range of not less than 10 nm and not more than 500 nm. The typical adjoining distance of the raised portions 34Bp, Dᵢₙₜ, is not less than 10 nm and not more than 1000 nm, and Dₚ<Dᵢₙₜ holds. That is, in the synthetic polymer film 34B, there is a flat portion between adjoining raised portions 34Bp. The raised portions 34Bp have the shape of a cylinder with a conical portion on the air side. The typical height of the raised portions 34Bp, Dₕ, is not less than 10 nm and not more than 500 nm. The raised portions 34Bp may be arranged regularly or may be arranged irregularly. When the raised portions 34Bp are arranged regularly, Dᵢₙₜ also represents the period of the arrangement. This also applies to the synthetic polymer film 34A, as a matter of course.

In this specification, the “moth-eye structure” includes not only surficial nanostructures that have an excellent antireflection function and that are formed by raised portions which have such a shape that the cross-sectional area (a cross section parallel to the film surface) increases, as in the raised portions 34Ap of the synthetic polymer film 34A shown in FIG. 2A, but also surficial nanostructures that are formed by raised portions which have a part where the cross-sectional area (a cross section parallel to the film surface) is constant, as in the raised portions 34Bp of the synthetic polymer film 34B shown in FIG. 2B. The tips of the raised portions do not need to be conical.

The plurality of raised portions of the solid surface illustrated in the examples have a generally-conical tip portion, although the shape of the plurality of raised portions is not limited to this shape. Note that, however, when the plurality of raised portions are formed using a mold, it is preferred from the viewpoint of mold releasability that the raised portions are tapered toward the tip end of the raised portions (the recessed portions of the mold are tapered toward the bottom of the recessed portions). The tip end does not need to be a pointed end. If the height of the raised portions (the depth of the recessed portions of the mold) exceeds 500 nm, disadvantageously, mold releasability will deteriorate, or manufacture of the mold will take time.

The surfaces of the synthetic polymer films 34A and 34B may be treated when necessary. For example, a water-repellent/oil-repellent agent or surface treatment agent may be applied to the surfaces in order to modify the surface tension (contact angle of drop). Some types of the water-repellent/oil-repellent agent or surface treatment agent lead to formation of a thin polymer film over the surfaces of the synthetic polymer films 34A and 34B. Alternatively, the surfaces of the synthetic polymer films 34A and 34B may be modified using plasma or the like. For example, by a plasma treatment, lipophilicity can be given to the surfaces of the synthetic polymer films 34A and 34B.

A mold for forming a moth-eye structure such as illustrated in FIG. 2A and FIG. 2B over the surface (hereinafter, referred to as “moth-eye mold”) has an inverted moth-eye structure obtained by inverting the moth-eye structure. Using an anodized porous alumina layer which has the inverted moth-eye structure as a mold without any modification enables inexpensive production of the moth-eye structure. Particularly when a moth-eye mold in the shape of a hollow cylinder is used, the moth-eye structure can be efficiently manufactured according to a roll-to-roll method. Such a moth-eye mold can be manufactured according to the methods disclosed in Patent Documents No. 5 to No. 8.

The manufacturing method of the moth-eye mold is not limited to the above-described method. A known nanostructure formation method, for example, various lithography methods such as interference lithography, electron beam lithography, etc., or a method of forming the structure by irradiating a glassy carbon substrate with an oxygen ion beam, can be used.

The influence of the interaction between the solid surface and cells (or the effect of the solid surface as a scaffold) on generation of a spheroid varies among different cell types and is not yet elucidated in many points before future research. However, as seen at least from the existing experimental results, a solid surface which has the features previously described in Items 2-9 can be suitably used in the drop culture method.

In the experimental examples which will be described later, a synthetic polymer film disclosed in Japanese Patent Application No. 2018-041073 (filing date: Mar. 7, 2018) and U.S. patent application Ser. No. 16/293,903 (claiming the priority of Japanese Patent Application No. 2018-041073) was used. The entire disclosures of the aforementioned patent applications are incorporated by reference in this specification. The synthetic polymer film disclosed in the aforementioned patent applications is characterized in that the pH of a liquid drop attached to the surface does not vary. Specifically, after the lapse of 5 minutes since placing a 200 μL drop of water on the surface of the synthetic polymer film, the pH of an aqueous solution can be not less than 6.5 and not more than 7.5. When the synthetic polymer film is made from a photocurable resin, an acid generated from a polymerization initiator is sometimes dissolved into the water attached to the surface. To prevent this, it is only necessary to use one or more polymerization initiators selected from the group consisting of, for example, ethanone, 1-[9-ethyl-6-(2-methylbenzoyl)-9H-carbazole-3-yl]-,1-(O-acetyloxime), 2-hydroxy-1-{4-[4-(2-hydroxy-2-methyl-propionyl)-benzyl]phenyl}-2-methyl-propan-1-one, and 1-[4-(2-hydroxyethoxy)-phenyl]-2-hydroxy-2-methyl-1-propane-1-one. Specifically, examples of such polymerization initiators include IRGACURE OXE02 (BASF), Omnirad 127 (IGM Resins), and Omnirad 2959 (IGM Resins).

If the variation of the pH of the liquid drop attached to the solid surface is excessively large, there is a probability that the growth rate of cells will decrease, the morphology of the spheroid will not be constant, or the reproducibility of tissue of the spheroid will decrease. From such viewpoints, the synthetic polymer film disclosed in the aforementioned patent applications is suitably used.

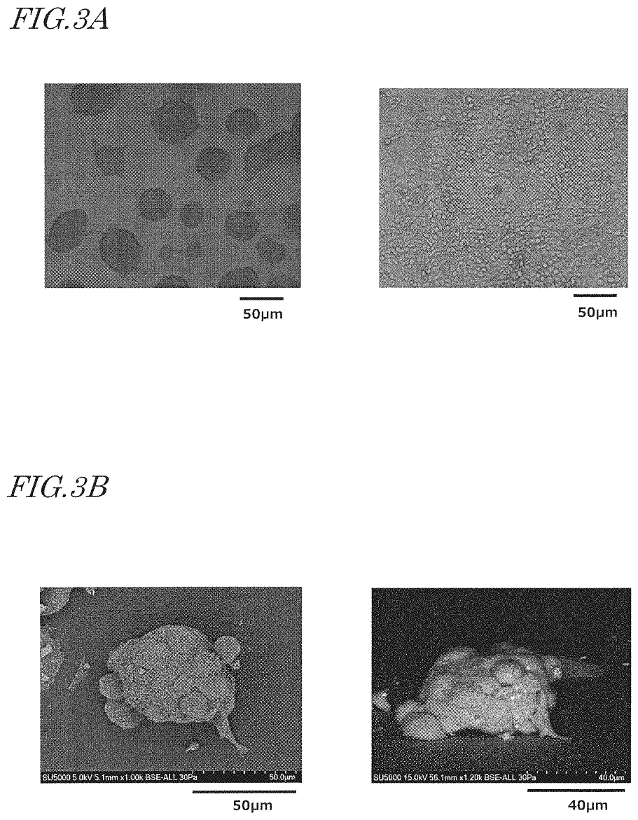

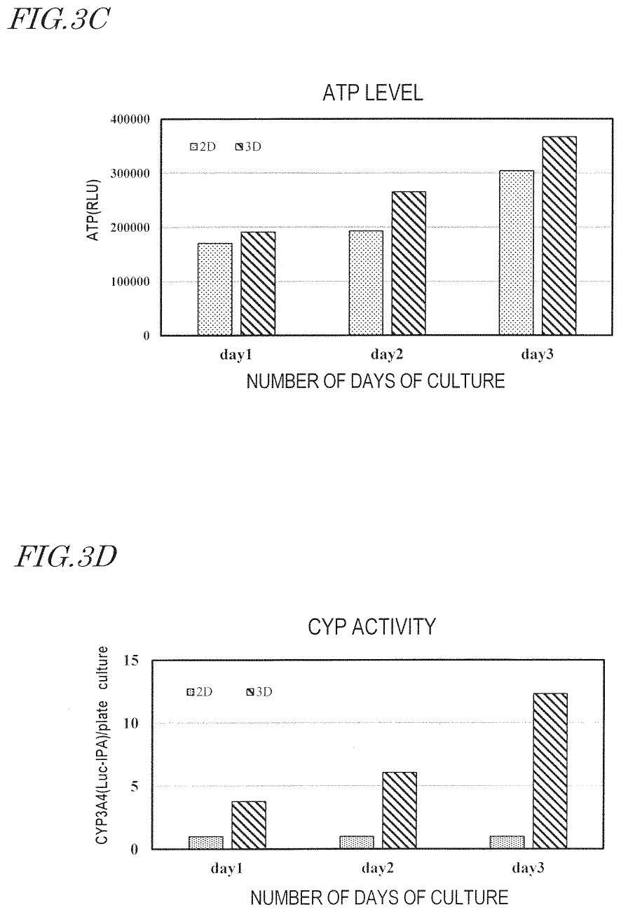

FIG. 3A shows images by an inverted phase contrast microscope of a result of drop culturing (left) and a result of two-dimensional culturing (right) of human liver cancer cell line HepG2 (left). FIG. 3B shows electron microscopic images of a HepG2 spheroid generated by a drop culture method. The left part of FIG. 3B is a top image. The right part of FIG. 3B is a side image.

The drop culturing was carried out according to the following method.

Firstly, the passage of human liver cancer cell line HepG2 cells was carried out in a petri dish for adherent culture of cells (for example, MS-11600 manufactured by Sumitomo Bakelite Co., Ltd.) under such atmospheric conditions that temperature: 37° C., carbon dioxide concentration: 5%, and relative humidity: 95%, using a culture medium which was prepared by adding 10% (final concentration) fetal bovine serum (FBS) to Dulbecco's modified eagle medium (D-MEM) which is an usual culture condition.

HepG2 cells in the process of passaging were dissociated from the culture dish using a trypsin reagent, which is a usual cell dissociation reagent, and the cell suspension was prepared such that the cell density counted using a fully automated cell counter while the cells were suspended in the culture medium at 1×10⁵ cells/mL. 25 μL of this cell suspension was measured out and attached to the surface of the photocured resin film which had the moth-eye structure so as to form a liquid drop. On the nano-convex film overlying a 35 mmφ dish, the optimum number of liquid drops was from 6 to 9. This liquid drop (25 μL) was cultured for 3 days under the same atmospheric conditions as those described above (temperature: 37° C., carbon dioxide concentration: 5%, relative humidity: 95%). Under these atmospheric conditions, the liquid drop maintained its shape even when a half or the whole of the culture medium was replaced for the purpose of adjusting the osmotic pressure in the culture medium.

As shown in the left part of FIG. 3A, when the drop culturing was carried out on the surface which had the moth-eye structure, spheroids were generated which had a generally-circular perimeter and which were three-dimensional. The generation of the three-dimensional spheroids can also be confirmed from the electron microscopic image of FIG. 3B.

The right part of FIG. 3A shows a result of usual two-dimensional culturing. In the right part of FIG. 3A, generation of a spheroid was not confirmed. Herein, the usual two-dimensional culturing was carried out as follows.

An appropriate amount (100 μL for 96-well plate; 2 mL for 35 mm dish) of a prepared cell suspension was seeded in a petri dish for culture with a usual cell adhesion surface coating (hydrophilic coating or the like), which was selected according to the capacity for the test, for example, MS-3096 or MS-11350 manufactured by Sumitomo Bakelite Co., Ltd., and culturing was carried out such that cells were able to grow in the form of a monolayer.

FIG. 3C shows the results of determination of the number of viable cells of liver cancer cell line HepG2 during drop culturing. Herein, it was quantified by measuring adenosine triphosphate (ATP), which is known to be the energy that is equal among viable cells from the same cell line. In FIG. 3C, 3D represents the result of the drop culture method, and 2D represents the result of the usual two-dimensional culture method.

As seen from FIG. 3C, the number of viable cells cultured by the drop culture method is generally equal to the number of viable cells cultured by the two-dimensional culture method. The number of viable cells cultured by a conventional three-dimensional culture method (for example, Patent Document No. 1) is smaller than the number of viable cells cultured by the two-dimensional culture method. If the number of viable cells cultured by the conventional three-dimensional culture method is 70% to 80% of the number of viable cells cultured by the two-dimensional culture method, it can be said that the cells are proliferating with high efficiency. Although the number of viable cells depends on the cell density, it was found that at a typical seeding density of 1×10⁵ cells/mL the drop culture method can achieve a generally equal cell proliferating rate to that achieved by the two-dimensional culture method.

FIG. 3D shows the results of evaluation of the reproducibility of tissue (or “gene expressivity”) of liver cancer cell HepG2 spheroids generated by the drop culture method. In FIG. 3D, 3D represents the result of the drop culture method, and 2D represents the result of the usual two-dimensional culture method.

Although HepG2 cells cultured by the two-dimensional culturing hardly sustain the hepatocellular functions, it is known that the three-dimensional culture enables coordination of cells according to the polarity and restores the activity of drug metabolism enzyme cytochrome P450 (hereinafter, also abbreviated as “CYP activity”), which is one of the characteristic liver functions. The CYP activity is used as one of the indices in evaluating the reproducibility of tissue of cultured spheroids. In view of such circumstances, the reproducibility of tissue of hepatocellular spheroids generated by the drop culture method was evaluated by measuring the P450 activity as described below.

The enzyme activity in cells was measured using a spheroid generated by the drop culture method, with the use of a P450-Glo™ Luciferin-IPA kit (manufactured by Promega Corporation), according to the instructions. For the sake of comparison, HepG2 cells were subjected to two-dimensional culturing such that the number of cells was equal to that of the cells in the liquid drop, and the P450 activity was measured according to the same method. Since it was expected that the cell growth rate was different between the two-dimensional culturing and the drop culturing, it is necessary to calculate the correction value for the enzyme activity per cell. Thus, for the purpose of measuring the number of viable cells in drop culturing or two-dimensional culturing under the same conditions as those used in measurement of the P450 enzyme activity, the quantification of ATP was carried out using a Cell Titer GloR kit (manufactured by Promega Corporation), and the RLU value was measured according to the instructions. The P450 enzyme activity in the two-dimensional culturing or drop culturing was divided by the ATP value. The relative P450 enzyme activity value of the HepG2 cells in drop culturing where the enzyme activity value per cell in the two-dimensional culturing was 1 was determined, which is represented by the graph shown in FIG. 3D.

As seen from FIG. 3D, in the HepG2 spheroid generated by the drop culture method, the CYP activity increased about ten times after culture of 3 days. It is estimated that the generated spheroid has high reproducibility of tissue.

It was concluded from the foregoing that a spheroid with high reproducibility of tissue can be produced by carrying out culture in a liquid drop formed on a solid surface.

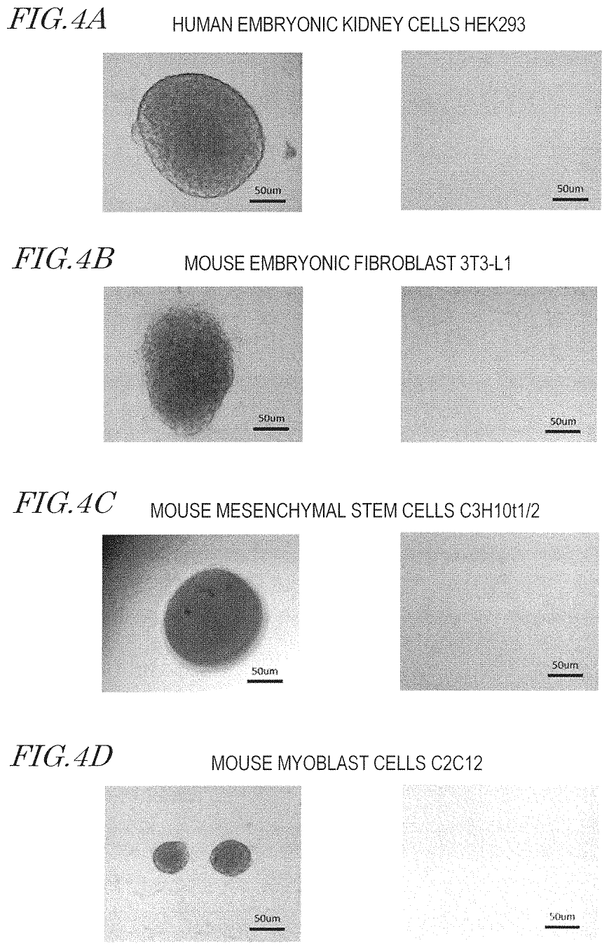

FIG. 4A, FIG. 4B, FIG. 4C and FIG. 4D show together the results of drop culturing (left) and the results of two-dimensional culturing (right) of various cells. FIG. 4A shows an optical microscopic image of a result of drop culturing (left) and an optical microscopic image of a result of two-dimensional culturing (right) of human embryonic kidney cells HEK293. FIG. 4B shows an optical microscopic image of a result of drop culturing (left) and an optical microscopic image of a result of two-dimensional culturing (right) of mouse embryonic fibroblast 3T3-L1. FIG. 4C shows an optical microscopic image of a result of drop culturing (left) and an optical microscopic image of a result of two-dimensional culturing (right) of mouse mesenchymal stem cells C3H10t1/2. FIG. 4D shows an optical microscopic image of a result of drop culturing (left) and an optical microscopic image of a result of two-dimensional culturing (right) of mouse myoblast cells C2C12.

As understood from the results of FIG. 4A, FIG. 4B, FIG. 4C and FIG. 4D, generation of spheroids by the drop culture method was confirmed, while no spheroids were formed by the two-dimensional culturing in either case. As understood from this, the drop culture method is suitably used in culturing of a wide variety of cell types.

Next, the results of research on the solid surface which is suitably used in the drop culture method are described.

[Synthetic Polymer Film]

Sample films which had the same configuration as the film 50A shown in FIG. 2A were produced using UV-curable resins of different compositions. The materials used in the UV-curable resins for production of the synthetic polymer films 34A of respective sample films are shown in TABLE 1. The compositions of UV-curable resins A, B and C are shown in TABLE 2. Each of the resins A, B and C contains a fluorine-based water-repellent/oil-repellent agent (water-repellent additive).

The mold used for forming the moth-eye structure over the surface was a porous alumina layer which was produced by the method disclosed in Patent Documents No. 5 to No. 8 and Japanese Patent Application No. 2018-041073. The flat “mold” used was alkali-free glass of 0.7 mm in thickness (EAGLE XG manufactured by CORNING).

In forming the synthetic polymer film 34A, the mold releasing treatments shown in TABLE 3 were performed on the respective molds. Three different treatments were performed using a fluorine-based mold releasing agent UD509 (OPTOOL UD509, modified perfluoropolyether, manufactured by DAIKIN INDUSTRIES, LTD.) at different concentrations.

As a parameter for characterizing the surfaces of the mold and the synthetic polymer film (the solid surface in the drop culture method), the contact angle was measured. TABLE 3 shows the contact angles of the surfaces of the molds. The contact angle of the solid surface with respect to the cell suspension affects the area in which the solid surface and cells are in contact (also referred to as “the bottom area of the liquid drop”) and the shape of the liquid drop. The shape of a spheroid to be produced varies depending on the contact angle, although it also depends on the cell type. Adjusting the contact angle according to the cell type is preferred.

The contact angle (static contact angle) was measured by a usual θ/2 method (half-angle Method: (θ/2=arctan(h/r), θ: contact angle, r: radius of liquid drop, h: height of liquid drop)). In measurement of the contact angle with the use of pure water, a 1 μL liquid drop, and liquid drops from 10 μL to 70 μL in consideration of the volume of a liquid drop used in the drop culture method, were used. In measurement of the contact angle with the use of a culture medium, liquid drops from 10 μL to 70 μL were used in consideration of the volume of a liquid drop used in the drop culture method. The contact angle changes over time. Therefore, a contact angle after the lapse of 1 second and a contact angle after the lapse of 10 seconds since placement of the liquid drop on the surface were measured. Herein, the contact angle for characterizing the solid surface refers to a static contact angle after the lapse of 10 seconds since placement of a liquid drop on the solid surface. Note that “Not Landed” means that the contact angle was 140° or more. Also, sliding angle measurements were taken by using liquid drops of 10 μL to 70 μL as were the contact angle measurements. A sliding angle refers to, when a surface on which a liquid drop has placed is inclined from the horizontal direction, a tilt angle at which the liquid drop begins to slide down.

The culture medium used was D-MEM (Low Glucose 1.0 g/L Glucose)/10% FBS. Note that the influence of the type and concentration of the culture medium on the contact angle and the sliding angle was within variations. Note also that the influence of adding cells to the culture medium on the contact angle was within variations.

TABLE 4 shows molds (type of mold releasing treatment) and the resin composition used for production of synthetic polymer films used in the experiment (Comparative Examples (Cx) 1 to 12 and Examples (Ex) 1 to 12), and the results of measurement of the contact angle of the surface of the respective synthetic polymer films with respect to water. The contact angle was measured using 1 μL of pure water. Contact angle measurements after the lapse of 1 second and contact angle measurements after the lapse of 10 seconds since placement of a liquid drop on the surface, and the difference (A) of the contact angle measurement after the lapse of 10 seconds from the contact angle measurement after the lapse of 1 second, are shown.

As seen from TABLE 4, a synthetic polymer film has higher water repellency when the synthetic polymer film is produced using a mold treated with a mold releasing treatment agent of higher concentration, although there are some variations. Comparing a flat surface and a surface with a moth-eye structure (hereinafter, referred to as “moth-eye surface”), the moth-eye surface has a greater contact angle and has higher water repellency (Lotus effect). At the moth-eye surfaces of Examples 1, 2 and 3, when a liquid drop formed at the tip of a needle in measurement of the contact angle was brought into contact with the moth-eye surface, the liquid drop did not attach to the moth-eye surface but stayed at the tip of the needle, so that measurement of the contact angle was impossible. When the contact angle generally exceeded 140°, the liquid drop failed to attach to the surface of the object as described herein.

As seen from the time change (A) of the contact angle, the time change of the contact angle was small and stable in each example except for Examples 10 and 11. It is estimated that the large time change of the contact angle in Examples 10 and 11 was attributed to the condition that the concentration of the mold releasing agent was low and the water-repellent/oil-repellent agent was not evenly or sufficiently attracted to the moth-eye surface. This is because the water-repellent/oil-repellent agent contained in the curable resin is attracted to the moth-eye surface due to the mold releasing treatment of the mold.

TABLE 5, TABLE 7, TABLE 9 and TABLE 11 show the results of measurement of the contact angle with respect to water and the contact angle with respect to the culture medium, with varying amounts of liquid drops (varying volumes of liquid drops). TABLE 5 (Comparative Examples 1-1 to 12-1 and Examples 1-1 to 12-1) shows the results obtained when a 10 μL liquid drop was used. TABLE 7 (Comparative Examples 1-2 to 12-2 and Examples 1-2 to 12-2) shows the results obtained when a 30 μL liquid drop was used. TABLE 9 (Comparative Examples 1-3 to 12-3 and Examples 1-3 to 12-3) shows the results obtained when a 50 μL liquid drop was used. TABLE 11 (Comparative Examples 1-4 to 12-4 and Examples 1-4 to 12-4) shows the results obtained when a 70 μL liquid drop was used.

Irrespective of whether water or the culture medium is used, the moth-eye surface has a greater contact angle and higher water repellency than the flat surface. As the volume of the liquid drop increases, the shape of the liquid drop becomes oblate under the influence of the gravity, and the contact angle decreases. This tendency was confirmed with water and the culture medium. Also, another tendency was confirmed that, when a synthetic polymer film is produced using a mold treated with a mold releasing treatment agent of higher concentration, the synthetic polymer film has higher water repellency, although there are some variations.

As the volume of the liquid drop increases, the sliding angle also decreases under the influence of the gravity. This tendency was confirmed with water and the culture medium. The moth-eye surface had a greater sliding angle than the flat surface. This is, in our estimation, attributed to the effect of minute raised portions of the moth-eye surface. That is, it is seen that the moth-eye surface has high water repellency and can maintain a high sliding angle.

The results of three-dimensional culture with the use of the surfaces of the respective synthetic polymer films are shown in TABLE 6 (Comparative Examples 1-1 to 12-1 and Examples 1-1 to 12-1), TABLE 8 (Comparative Examples 1-2 to 12-2 and Examples 1-2 to 12-2), TABLE 10 (Comparative Examples 1-3 to 12-3 and Examples 1-3 to 12-3), and TABLE 12 (Comparative Examples 1-4 to 12-4 and Examples 1-4 to 12-4).

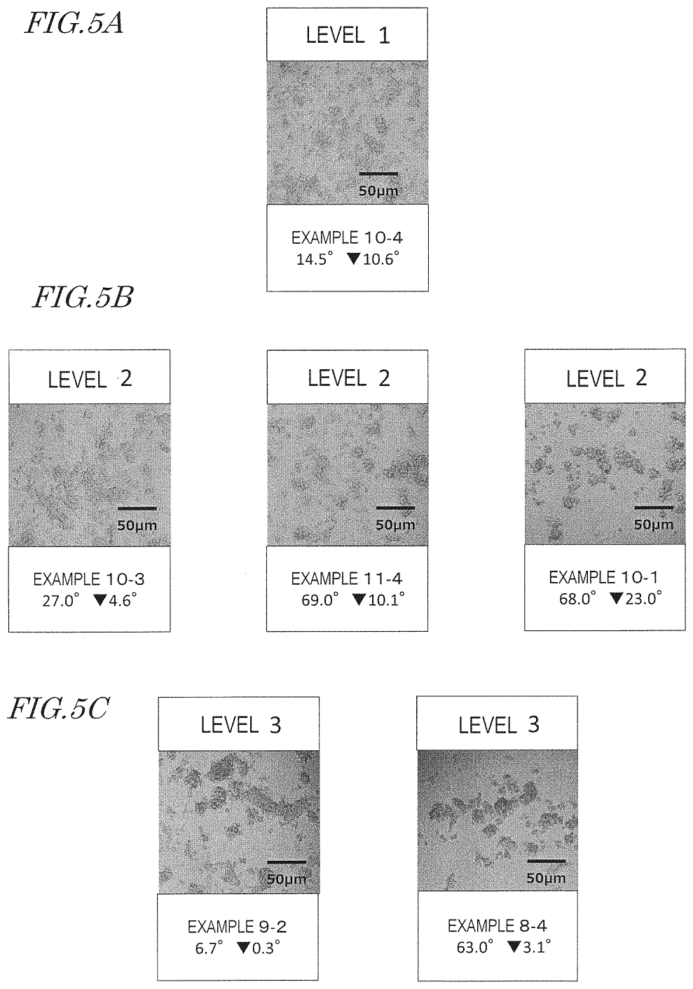

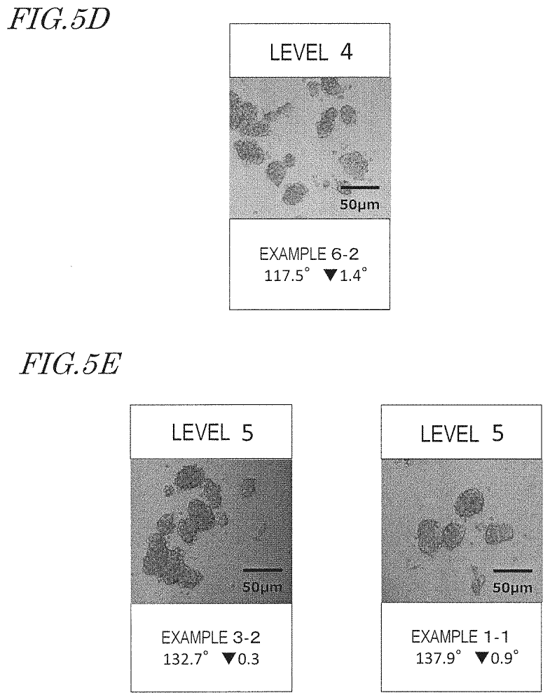

Evaluation of spheroidization was made by observation of the morphology with the use of an optical microscope. The level of spheroidization was put to five-grade evaluation where a greater level number means a better state of spheroidization (aggregated at high density). Examples of the results of the morphology observation with the use of an optical microscope are shown in FIG. 5A (Level 1), FIG. 5B (Level 2), FIG. 5C (Level 3), FIG. 5D (Level 4) and FIG. 5E (Level 5). In TABLE 6, TABLE 8, TABLE 10 and TABLE 12, ◯ (good) represents generation of a spheroid at Level 3 or higher, Δ (tolerable) represents generation of a spheroid at Level 2 or Level 1, and x (not tolerable) represents that generation of a spheroid was not found. FIG. 5A shows an optical microscopic image of spheroids of Example 10-4. FIG. 5B shows optical microscopic images of spheroids of Example 10-3 (left), Example 11-4 (middle), and Example 10-1 (right). FIG. 5C shows optical microscopic images of spheroids of Example 9-2 (left) and Example 8-4 (right). FIG. 5D shows an optical microscopic image of spheroids of Example 6-2. FIG. 5E shows optical microscopic images of spheroids of Example 3-(left) and Example 1-1 (right). The optical microscopic images are each accompanied by the contact angle of the culture medium (after the lapse of 10 seconds) and the variation of the contact angle (▾ represents minus).

Operational easiness in replacing the culture medium was evaluated by the contact angle. When the contact angle was not less than 110°, the operational easiness was judged as ⊚ (excellent). When the contact angle was not less than 90° and less than 110°, the operational easiness was judged as ◯ (good). When the contact angle was less than 90°, the operational easiness was judged as Δ (tolerable). When the height of the liquid drop was less than 1 mm, the operational easiness in replacing the culture medium decreased and, therefore, the operational easiness was judged as x (not tolerable). Since the drop culture method provides high viability, some cell types can have a long culture duration (more than several days). In such a case, the culture medium in the liquid drop reduces through evaporation. Also, the waste product in the liquid drop increases. In view of such, the step of adding the culture medium into the liquid drop and, furthermore, the step of aspirating part of the culture medium from the liquid drop before adding the culture medium are preferably performed. To efficiently perform such an operation of replacing the culture medium with the use of a dispenser, the height of the liquid drop is preferably not less than 1 mm. The contact angle determined by the θ/2 method is based on the assumption that the shape of the liquid drop is a part of a circle (θ/2=arctan(h/r), θ: contact angle, r: radius of liquid drop, h: height of liquid drop). In view of this relationship, for example, when the volume of the liquid drop is 70 μL, and when the contact angle is 17°, the height h is 1 mm (when the volume of the liquid drop is 50 μL, the contact angle is 20°; when the volume of the liquid drop is 30 μL, the contact angle is 26°; when the volume of the liquid drop is 10 μL, the contact angle is 44°; in each case, the height h is 1 mm). Thus, only Example 10-4 (the contact angle of the culture medium after the lapse of 10 seconds was 14.5°, i.e., less than 17°) was judged as x.

As for handling easiness, the capability of stably holding the liquid drop on the solid surface during operations was evaluated by the sliding angle. The liquid drop on the solid surface sometimes moves (slides or rolls) when the solid surface tilts or vibrates. To prevent the movement of the liquid drop, it is necessary to keep the solid surface from tilting or vibrating during culturing and, accordingly, the operational easiness deteriorates. For example, the sliding angle of the solid surface with respect to the culture medium is set to 45° or greater, whereby the liquid drop can be relatively stably held on the solid surface. The handling easiness was judged as ⊚ (excellent) when the sliding angle was not less than 90°, ◯ (good) when the sliding angle was not less than 45° and less than 90°, Δ (tolerable) when the sliding angle was not less than 10° and less than 45°, and x (not tolerable) when the sliding angle was less than 10°.

As seen from the evaluation results of spheroidization in TABLE 6, TABLE 8, TABLE 10 and TABLE 12, generation of a spheroid was confirmed in all of Examples where the moth-eye surface was used, while generation of a spheroid was not confirmed in any of Comparative Examples where the flat surface was used. In Examples, as seen from FIG. 5A to FIG. 5E, there is such a tendency that, as the contact angle increases, a spheroid in a better state is produced. This is because, in our estimation, as the contact angle increases, the shape of the liquid drop is closer to a sphere, and cells are aggregated with high density at the bottom surface of the liquid drop. The contact angle is preferably at least not less than 17°, more preferably not less than 90°. It is required that the value of the contact angle after the lapse of 10 seconds since placement of the liquid drop meets the above-described conditions.

As seen from the comparison of Example 11-4 (middle of FIG. 5B) and Example 10-1 (right of FIG. 5B) with Example 8-4 (right of FIG. 5C), there is such a tendency that, as the difference A in contact angle decreases, a spheroid in a better state is generated. This is because, in our estimation, as the variation in contact angle in the period of 10 seconds after placement of the liquid drop decreased, the shape of the liquid drop in a culturing period was more likely to be maintained (unlikely to be oblate) and, as a result, the cell aggregation effect which was attributed to the shape of the liquid drop was greater.

From the viewpoint of formation of a liquid drop and manageability, the volume of the liquid drop is preferably not less than 10 μL and not more than 50 μL (see TABLE 6, TABLE 8 and TABLE 10, particularly Example 1 to Example 6).

The seeding density of cells contained in the liquid drop is, for example, not less than 10³ cells/mL and not more than 10⁷ cells/mL. One of the advantages of the drop culture method is the capability of accurately controlling the number of cells contained in the liquid drop. The number of cells is typically within the above-described range but can be suitably adjusted according to the cell type or the volume of the liquid drop.

As described above, from the viewpoint of handling easiness, the sliding angle of the liquid drop is preferably not less than 45°, more preferably not less than 90°. The sliding angle may be evaluated based on a value after the lapse of 20 seconds since placement of the drop.

TABLE 1

| Materials | | Product Name | Manufacturer Name | Compound Name |
|---|---|---|---|---|
| Monomer | M280 | M280 | MIWON | polyethylene glycol (400) diacrylate |
| | M282 | M282 | MIWON | polyethylene glycol (200) diacrylate |
| | ACMO | ACMO ® | KJ Chemicals Corporation | N,N-acryloylmorpholine |
| Mold | MT70 | FOMBLIN ® MT70 | SOLVAY | perfluoropolyether derivative; 80% |
| Releasing | | | | methyl ethyl ketone (solvent); 20% |
| Agent | FAAC6 | CHEMINOX | Unimatec Corporation | 2-(perfluorohexyl)ethyl acrylate |
| | | FAAC-6 | | |
| | KY1203E | X71-KY1203E | Shin-Etsu Silicone | fluorine-containing acrylic compound; 20% |
| | | | | methyl ethyl ketone (solvent); 80% |
| Polymeriza- | OXE02 | IRGACURE | BASF | ethanone,1-[9-ethyl-6-(2-methylbenzoyl)- |
| tion Initiator | | OXE02 | | 9H-carbazol-3-yl]-,1-(O-acetyloxime) |

TABLE 2

| | Monomer | | | Initiator | Water-repellent/oil-repellent Agent | | |
|---|---|---|---|---|---|---|---|
| | M280 | M282 | ACMO | OXE02 | MT70 | FAAC6 | KY1203E |
| Resin A | 28.6% | 63.8% | 2.9% | 1.0% | — | — | 3.8% |
| Resin B | 29.1% | 65.0% | 2.9% | 1.0% | 1.9% | — | — |
| Resin C | 27.5% | 61.5% | 2.8% | 0.9% | 3.7% | 3.7% | — |

TABLE 3

| | Mold Releasing | | | |
|---|---|---|---|---|
| | Treatment | | | |
| | Mold Releasing | Water Contact Angle (°) 1 μL | | |
| | Agent (UD509) | After lapse of 1 second | After lapse of 10 seconds | |
| Mold | Concentration | since placement of drop | since placement of drop | [symbol] |
| flat 1 | 0.1% | 105.05 | 104.85 | −0.2 |
| flat 2 | 0.001% | 100.1 | 99.05 | −1.05 |
| flat 3 | 0.0001% | 72.35 | 69.55 | −2.8 |
| flat 4 | 0.00001% | 59.25 | 58.35 | −0.9 |
| moth-eye 1 | 0.1% | Not Landed (>140) | | |
| moth-eye 2 | 0.001% | 128.5 | 128.05 | −0.45 |
| moth-eye 3 | 0.0001% | 124.6 | 124.55 | −0.05 |
| moth-eye 4 | 0.00001% | 124.3 | 122.85 | −1.45 |

TABLE 4

| | | | Water Contact Angle (°) 1 μL | | |
|---|---|---|---|---|---|
| | | | After lapse | After lapse | |
| | Mold | Resin | of 1 second | of 10 seconds | [symbol] |
| Cx. 1 | flat 1 | A | 105.8 | 105.4 | −0.4 |
| Cx. 2 | flat 1 | B | 108.1 | 107.0 | −1.1 |
| Cx. 3 | flat 1 | C | 107.3 | 107.8 | 0.5 |
| Cx. 4 | flat 2 | A | 96.1 | 96.9 | 0.8 |
| Cx. 5 | flat 2 | B | 98.2 | 95.6 | −2.6 |
| Cx. 6 | flat 2 | C | 104.1 | 103.4 | −0.7 |
| Cx. 7 | flat 3 | A | 77.4 | 75.1 | −2.3 |
| Cx. 8 | flat 3 | B | 82.1 | 77.2 | −4.9 |
| Cx. 9 | flat 3 | C | 86.9 | 82.0 | −4.9 |
| Cx. 10 | flat 4 | A | 64.0 | 61.5 | −2.6 |
| Cx. 11 | flat 4 | B | 59.8 | 57.8 | −2.0 |
| Cx. 12 | flat 4 | C | 70.9 | 66.6 | −4.3 |
| Ex. 1 | moth-eye 1 | A | Not Landed (>140) | | |
| Ex. 2 | moth-eye 1 | B | Not Landed (>140) | | |
| Ex. 3 | moth-eye 1 | C | Not Landed (>140) | | |
| Ex. 4 | moth-eye 2 | A | 131.0 | 131.1 | 0.1 |
| Ex. 5 | moth-eye 2 | B | 134.3 | 134.5 | 0.2 |
| Ex. 6 | moth-eye 2 | C | 138.5 | 138.6 | 0.1 |
| Ex. 7 | moth-eye 3 | A | 113.2 | 107.5 | −5.7 |
| Ex. 8 | moth-eye 3 | B | 112.3 | 109.9 | −2.3 |
| Ex. 9 | moth-eye 3 | C | 133.5 | 132.2 | −1.3 |
| Ex. 10 | moth-eye 4 | A | 108.0 | 91.2 | −16.8 |
| Ex. 11 | moth-eye 4 | B | 94.6 | 63.8 | −30.8 |
| Ex. 12 | moth-eye 4 | C | 120.0 | 118.0 | −2.1 |

TABLE 5

| | | | Water | | | | Culture Medium (LG/F) | | | |
|---|---|---|---|---|---|---|---|---|---|---|
| | | | Contact | Contact | | | Contact | Contact | | |
| | | | Angle | Angle | | | Angle | Angle | | |
| | | | (°) | (°) | | | (°) | (°) | | |
| Liquid | | | After | After | [symbol] | | After | After | [symbol] | |
| Drop | | | lapse of | lapse of | Contact | Sliding | lapse of | lapse of | Contact | Sliding |
| Amount | | | 1 sec- | 10 sec- | Angle | Angle | 1 sec- | 10 sec- | Angle | Angle |
| 10 μL | Mold | Resin | ond | onds | (°) | (°) | ond | onds | (°) | (°) |
| Cx. 1-1 | flat 1 | A | 67.8 | 66.7 | −1.1 | 90.0 | 98.8 | 98.6 | −0.2 | 63.1 |
| Cx. 2-1 | flat 1 | B | 99.7 | 99.3 | −0.4 | 60.1 | 98.2 | 97.6 | −0.6 | 65.5 |
| Cx. 3-1 | flat 1 | C | 83.5 | 82.4 | −1.1 | 78.5 | 95.6 | 95.6 | 0.0 | 64.0 |
| Cx. 4-1 | flat 2 | A | 81.1 | 77.7 | −3.4 | 90.0 | 78.0 | 74.8 | −3.2 | 90.0 |
| Cx. 5-1 | flat 2 | B | 101.6 | 100.6 | −1.0 | 58.0 | 98.1 | 97.6 | −0.5 | 75.3 |
| Cx. 6-1 | flat 2 | C | 98.4 | 98.1 | −0.3 | 78.0 | 96.1 | 95.1 | −1.0 | 90.0 |
| Cx. 7-1 | flat 3 | A | 61.7 | 60.6 | −1.1 | 90.0 | 57.2 | 56.6 | −0.6 | 90.0 |
| Cx. 8-1 | flat 3 | B | 80.3 | 78.0 | −2.3 | 90.0 | 76.3 | 73.8 | −2.5 | 90.0 |
| Cx. 9-1 | flat 3 | C | 80.5 | 78.6 | −1.9 | 90.0 | 75.4 | 73.7 | −1.7 | 90.0 |
| Cx. 10-1 | flat 4 | A | 69.5 | 66.3 | −3.2 | 90.0 | 64.0 | 61.8 | −2.2 | 90.0 |
| Cx. 11-1 | flat 4 | B | 71.1 | 70.1 | −1.0 | 90.0 | 66.1 | 64.6 | −1.5 | 90.0 |
| Cx. 12-1 | flat 4 | C | 72.2 | 73.3 | 1.1 | 90.0 | 70.2 | 70.8 | 0.6 | 90.0 |
| Ex. 1-1 | moth-eye 1 | A | Not Landed (>140) | | | 5° or | 137.0 | 137.9 | 0.9 | 90.0 |
| | | | | | | less | | | | |
| Ex. 2-1 | moth-eye 1 | B | Not Landed (>140) | | | 5° or | 137.1 | 136.9 | −0.2 | 90.0 |
| | | | | | | less | | | | |
| Ex. 3-1 | moth-eye 1 | C | Not Landed (>140) | | | 5° or | 137.5 | 137.9 | 0.4 | 90.0 |
| | | | | | | less | | | | |
| Ex. 4-1 | moth-eye 2 | A | 138.6 | 138.4 | −0.2 | 90.0 | 120.1 | 116.6 | −3.5 | 90.0 |
| Ex. 5-1 | moth-eye 2 | B | 136.5 | 136.5 | 0.0 | 90.0 | 131.1 | 128.1 | −3.0 | 90.0 |
| Ex. 6-1 | moth-eye 2 | C | 135.8 | 134.6 | −1.2 | 90.0 | 132.7 | 128.6 | −4.1 | 90.0 |
| Ex. 7-1 | moth-eye 3 | A | 117.2 | 117.5 | 0.3 | 90.0 | 103.2 | 94.1 | −9.1 | 90.0 |
| Ex. 8-1 | moth-eye 3 | B | 129.0 | 128.6 | −0.4 | 90.0 | 127.8 | 121.8 | −6.0 | 90.0 |
| Ex. 9-1 | moth-eye 3 | C | 133.3 | 132.5 | −0.8 | 90.0 | 123.7 | 122.3 | −1.4 | 90.0 |
| Ex. 10-1 | moth-eye 4 | A | 114.1 | 112.1 | −2.0 | 90.0 | 91.0 | 68.0 | −23.0 | 90.0 |
| Ex. 11-1 | moth-eye 4 | B | 121.9 | 121.8 | −0.1 | 90.0 | 115.4 | 112.7 | −2.7 | 90.0 |
| Ex. 12-1 | moth-eye 4 | C | 131.4 | 131.0 | −0.4 | 90.0 | 131.5 | 128.2 | −3.3 | 90.0 |

TABLE 6

| | | Operational | |
|---|---|---|---|
| | | easiness in re- | |
| Liquid Drop Amount | | placing culture | Handling |
| 10 μL | Spheroidization | medium | easiness |
| Cx. 1-1 | X | ◯ | ◯ |
| Cx. 2-1 | X | ◯ | ◯ |
| Cx. 3-1 | X | ◯ | ◯ |
| Cx. 4-1 | X | Δ | ⊚ |
| Cx. 5-1 | X | ◯ | ◯ |
| Cx. 6-1 | X | ◯ | ⊚ |
| Cx. 7-1 | X | Δ | ⊚ |
| Cx. 8-1 | X | Δ | ⊚ |
| Cx. 9-1 | X | Δ | ⊚ |
| Cx. 10-1 | X | Δ | ⊚ |
| Cx. 11-1 | X | Δ | ⊚ |
| Cx. 12-1 | X | Δ | ⊚ |
| Ex. 1-1 | ◯ | ⊚ | ⊚ |
| Ex. 2-1 | ◯ | ⊚ | ⊚ |
| Ex. 3-1 | ◯ | ⊚ | ⊚ |
| Ex. 4-1 | ◯ | ⊚ | ⊚ |
| Ex. 5-1 | ◯ | ⊚ | ⊚ |
| Ex. 6-1 | ◯ | ⊚ | ⊚ |
| Ex. 7-1 | ◯ | ◯ | ⊚ |
| Ex. 8-1 | ◯ | ⊚ | ⊚ |
| Ex. 9-1 | ◯ | ⊚ | ⊚ |
| Ex. 10-1 | Δ | Δ | ⊚ |
| Ex. 11-1 | ◯ | ⊚ | ⊚ |
| Ex. 12-1 | ◯ | ⊚ | ⊚ |

TABLE 7

| | Water | | | | Culture Medium (LG/F) | | | |
|---|---|---|---|---|---|---|---|---|
| | Contact | Contact | | | Contact | Contact | | |
| | Angle | Angle | | | Angle | Angle | | |
| | (°) | (°) | | | (°) | (°) | | |
| Liquid | After | After | | | After | After | | |
| Drop | lapse of | lapse of | [symbol] Contact | Sliding | lapse of | lapse of | [symbol] Contact | Sliding |
| Amount | 1 sec- | 10 sec- | Angle | Angle | 1 sec- | 10 sec- | Angle | Angle |
| 30 μL | ond | onds | (°) | (°) | ond | onds | (°) | (°) |
| Cx. 1-2 | 59.9 | 60.5 | 0.6 | 67.9 | 78.3 | 77.6 | −0.7 | 61.2 |
| Cx. 2-2 | 78.1 | 81.9 | 3.8 | 29.5 | 77.8 | 77.8 | 0.0 | 43.1 |
| Cx. 3-2 | 74.9 | 74.5 | −0.4 | 32.6 | 82.5 | 81.0 | −1.5 | 50.3 |
| Cx. 4-2 | 69.3 | 68.8 | −0.5 | 71.0 | 66.0 | 65.9 | −0.1 | 65.3 |
| Cx. 5-2 | 78.2 | 79.1 | 0.9 | 51.0 | 74.7 | 75.9 | 1.2 | 45.0 |
| Cx. 6-2 | 78.2 | 78.7 | 0.5 | 33.0 | 76.2 | 75.2 | −1.0 | 40.2 |
| Cx. 7-2 | 57.0 | 57.8 | 0.8 | 75.1 | 52.8 | 53.3 | 0.5 | 70.3 |
| Cx. 8-2 | 65.6 | 65.6 | 0.0 | 72.0 | 61.1 | 61.4 | 0.3 | 67.5 |
| Cx. 9-2 | 66.4 | 66.5 | 0.1 | 80.0 | 61.3 | 61.0 | −0.3 | 73.6 |
| Cx. 10-2 | 59.3 | 59.5 | 0.2 | 72.2 | 57.2 | 55.8 | −1.4 | 65.6 |
| Cx. 11-2 | 59.8 | 59.2 | −0.6 | 61.9 | 54.8 | 53.7 | −1.1 | 69.5 |
| Cx. 12-2 | 60.3 | 58.7 | −1.6 | 73.7 | 56.8 | 54.2 | −2.6 | 68.2 |
| Ex. 1-2 | Not Landed (>140) | | | 5° or | 115.4 | 116.3 | 0.9 | 90.0 |
| | | | | less | | | | |
| Ex. 2-2 | Not Landed (>140) | | | 5° or | 125.8 | 125.0 | −0.9 | 90.0 |
| | | | | less | | | | |
| Ex. 3-2 | Not Landed (>140) | | | 5° or | 133.0 | 132.7 | −0.3 | 90.0 |
| | | | | less | | | | |
| Ex. 4-2 | 129.3 | 129.9 | 0.6 | 90.0 | 114.6 | 113.6 | −1.0 | 90.0 |
| Ex. 5-2 | 127.3 | 126.8 | −0.5 | 90.0 | 131.0 | 128.7 | −2.3 | 90.0 |
| Ex. 6-2 | 124.5 | 123.6 | −0.9 | 90.0 | 118.8 | 117.5 | −1.4 | 90.0 |
| Ex. 7-2 | 92.1 | 91.8 | −0.3 | 90.0 | 93.6 | 86.9 | −6.7 | 90.0 |
| Ex. 8-2 | 103.5 | 102.4 | −1.1 | 90.0 | 109.1 | 107.8 | −1.3 | 90.0 |
| Ex. 9-2 | 119.2 | 119.8 | 0.6 | 90.0 | 87.0 | 86.7 | −0.3 | 90.0 |
| Ex. 10-2 | 87.4 | 87.4 | 0.0 | 90.0 | 56.5 | 41.9 | −14.6 | 90.0 |
| Ex. 11-2 | 100.2 | 100.7 | 0.5 | 90.0 | 102.0 | 99.1 | −2.9 | 90.0 |
| Ex. 12-2 | 103.2 | 103.5 | 0.3 | 90.0 | 112.7 | 112.0 | −0.7 | 90.0 |

TABLE 8

| | | Operational | |
|---|---|---|---|
| | | easiness in re- | |
| Liquid Drop Amount | | placing culture | Handling |
| 30 μL | Spheroidization | medium | easiness |
| Cx. 1-2 | X | Δ | ◯ |
| Cx. 2-2 | X | Δ | Δ |
| Cx. 3-2 | X | Δ | ◯ |
| Cx. 4-2 | X | Δ | ◯ |
| Cx. 5-2 | X | Δ | ◯ |
| Cx. 6-2 | X | Δ | Δ |
| Cx. 7-2 | X | Δ | ◯ |
| Cx. 8-2 | X | Δ | ◯ |
| Cx. 9-2 | X | Δ | ◯ |
| Cx. 10-2 | X | Δ | ◯ |
| Cx. 11-2 | X | Δ | ◯ |
| Cx. 12-2 | X | Δ | ◯ |
| Ex. 1-2 | ◯ | ⊚ | ⊚ |
| Ex. 2-2 | ◯ | ⊚ | ⊚ |
| Ex. 3-2 | ◯ | ⊚ | ⊚ |
| Ex. 4-2 | ◯ | ⊚ | ⊚ |
| Ex. 5-2 | ◯ | ⊚ | ⊚ |
| Ex. 6-2 | ◯ | ⊚ | ⊚ |
| Ex. 7-2 | ◯ | Δ | ⊚ |
| Ex. 8-2 | ◯ | ◯ | ⊚ |
| Ex. 9-2 | ◯ | Δ | ⊚ |
| Ex. 10-2 | Δ | Δ | ⊚ |
| Ex. 11-2 | ◯ | ◯ | ◯ |
| Ex. 12-2 | ◯ | ⊚ | ⊚ |

TABLE 9

| | Water | | | | Culture Medium (LG/F) | | | |
|---|---|---|---|---|---|---|---|---|
| | Contact | Contact | | | Contact | Contact | | |
| | Angle | Angle | | | Angle | Angle | | |
| | (°) | (°) | | | (°) | (°) | | |
| Liquid | After | After | | | After | After | | |
| Drop | lapse of | lapse of | [symbol] Contact | Sliding | lapse of | lapse of | [symbol] Contact | Sliding |
| Amount | 1 sec- | 10 sec- | Angle | Angle | 1 sec- | 10 sec- | Angle | Angle |
| 50 μL | ond | onds | (°) | (°) | ond | onds | (°) | (°) |
| Cx. 1-3 | 57.7 | 57.2 | −0.5 | 35.0 | 77.3 | 78.5 | 1.2 | 51.7 |
| Cx. 2-3 | 85.3 | 83.9 | −1.4 | 21.8 | 75.5 | 75.4 | −0.1 | 43.1 |
| Cx. 3-3 | 71.8 | 72.2 | 0.4 | 25.9 | 84.4 | 83.5 | −0.9 | 48.0 |
| Cx. 4-3 | 75.6 | 73.6 | −2.0 | 48.2 | 70.6 | 68.1 | −2.5 | 55.1 |
| Cx. 5-3 | 77.5 | 75.8 | −1.7 | 50.9 | 72.2 | 70.2 | −2.0 | 52.5 |
| Cx. 6-3 | 77.1 | 76.9 | −0.2 | 57.7 | 72.6 | 71.7 | −0.8 | 51.3 |
| Cx. 7-3 | 57.0 | 56.1 | −0.9 | 48.9 | 51.9 | 50.7 | −1.2 | 58.6 |
| Cx. 8-3 | 62.3 | 62.3 | 0.0 | 52.5 | 57.3 | 57.0 | −0.3 | 54.7 |
| Cx. 9-3 | 62.7 | 63.2 | 0.5 | 57.1 | 58.2 | 57.7 | −0.5 | 55.4 |
| Cx. 10-3 | 55.8 | 55.6 | −0.2 | 47.3 | 54.5 | 53.2 | −1.3 | 35.1 |
| Cx. 11-3 | 56.4 | 56.4 | 0.0 | 46.6 | 51.3 | 51.1 | −0.2 | 48.3 |
| Cx. 12-3 | 57.7 | 57.8 | 0.1 | 44.1 | 52.3 | 52.2 | −0.1 | 46.9 |
| Ex. 1-3 | Not Landed (>140) | | | 5° or | 112.3 | 112.1 | −0.2 | 47.0 |
| | | | | less | | | | |
| Ex. 2-3 | Not Landed (>140) | | | 5° or | 125.4 | 124.6 | −0.8 | 36.5 |
| | | | | less | | | | |
| Ex. 3-3 | Not Landed (>140) | | | 5° or | 128.5 | 128.1 | −0.4 | 26.5 |
| | | | | less | | | | |
| Ex. 4-3 | 123.0 | 123.2 | 0.2 | 50.6 | 117.8 | 117.4 | −0.4 | 90.0 |
| Ex. 5-3 | 117.5 | 117.6 | 0.1 | 46.6 | 111.7 | 111.6 | −0.1 | 90.0 |
| Ex. 6-3 | 117.5 | 117.7 | 0.2 | 48.6 | 112.5 | 112.3 | −0.2 | 39.0 |
| Ex. 7-3 | 89.5 | 89.5 | 0.0 | 90.0 | 83.7 | 79.8 | −3.9 | 90.0 |
| Ex. 8-3 | 97.9 | 97.8 | −0.1 | 90.0 | 104.6 | 102.0 | −2.6 | 70.6 |
| Ex. 9-3 | 115.8 | 115.6 | −0.2 | 68.3 | 98.7 | 96.8 | −1.9 | 61.5 |
| Ex. 10-3 | 86.9 | 86.9 | 0.0 | 90.0 | 31.6 | 27.0 | −4.6 | 90.0 |
| Ex. 11-3 | 94.2 | 94.2 | 0.0 | 90.0 | 96.7 | 93.2 | −3.5 | 90.0 |
| Ex. 12-3 | 123.0 | 123.2 | 0.2 | 50.6 | 117.8 | 117.4 | −0.4 | 90.0 |

TABLE 10

| | | Operational | |
|---|---|---|---|
| | | easiness in re- | |
| Liquid Drop Amount | | placing culture | Handling |
| 50 μL | Spheroidization | medium | easiness |
| Cx. 1-3 | X | Δ | ◯ |
| Cx. 2-3 | X | Δ | Δ |
| Cx. 3-3 | X | Δ | ◯ |
| Cx. 4-3 | X | Δ | ◯ |
| Cx. 5-3 | X | Δ | ◯ |
| Cx. 6-3 | X | Δ | ◯ |
| Cx. 7-3 | X | Δ | ◯ |
| Cx. 8-3 | X | Δ | ◯ |
| Cx. 9-3 | X | Δ | ◯ |
| Cx. 10-3 | X | Δ | Δ |
| Cx. 11-3 | X | Δ | ◯ |
| Cx. 12-3 | X | Δ | ◯ |
| Ex. 1-3 | ◯ | ⊚ | ◯ |
| Ex. 2-3 | ◯ | ⊚ | Δ |
| Ex. 3-3 | ◯ | ⊚ | Δ |
| Ex. 4-3 | ◯ | ⊚ | ⊚ |
| Ex. 5-3 | ◯ | ⊚ | ⊚ |
| Ex. 6-3 | ◯ | ⊚ | Δ |
| Ex. 7-3 | ◯ | Δ | ⊚ |
| Ex. 8-3 | ◯ | ◯ | ◯ |
| Ex. 9-3 | ◯ | ◯ | ◯ |
| Ex. 10-3 | Δ | Δ | ⊚ |
| Ex. 11-3 | ◯ | ◯ | ⊚ |
| Ex. 12-3 | ◯ | ◯ | ◯ |

TABLE 11

| | Water | | | | Culture Medium (LG/F) | | | |
|---|---|---|---|---|---|---|---|---|
| | Contact | Contact | | | Contact | Contact | | |
| | Angle | Angle | | | Angle | Angle | | |
| | (°) | (°) | | | (°) | (°) | | |
| Liquid | After | After | | | After | After | | |
| Drop | lapse of | lapse of | [symbol] Contact | Sliding | lapse of | lapse of | [symbol] Contact | Sliding |
| Amount | 1 sec- | 10 sec- | Angle | Angle | 1 sec- | 10 sec- | Angle | Angle |
| 70 μL | ond | onds | (°) | (°) | ond | onds | (°) | (°) |
| Cx. 1-4 | 88.6 | 88.5 | −0.1 | 18.7 | 84.1 | 83.5 | −0.6 | 13.2 |
| Cx. 2-4 | 89.3 | 89.5 | 0.2 | 18.3 | 84.5 | 84.2 | −0.3 | 12.7 |
| Cx. 3-4 | 87.4 | 87.6 | 0.2 | 24.0 | 82.2 | 82.5 | 0.3 | 18.6 |
| Cx. 4-4 | 72.0 | 72.0 | 0.0 | 37.1 | 66.9 | 66.6 | −0.3 | 32.0 |
| Cx. 5-4 | 73.2 | 74.7 | 1.5 | 36.2 | 68.7 | 68.0 | −0.7 | 31.2 |
| Cx. 6-4 | 73.9 | 73.9 | 0.0 | 44.8 | 68.7 | 68.1 | −0.6 | 39.5 |
| Cx. 7-4 | 56.6 | 56.4 | −0.2 | 39.3 | 51.3 | 50.7 | −0.6 | 33.8 |
| Cx. 8-4 | 58.5 | 58.7 | 0.2 | 40.0 | 53.4 | 53.2 | −0.2 | 34.9 |
| Cx. 9-4 | 61.9 | 61.8 | −0.1 | 44.1 | 56.5 | 56.5 | 0.0 | 38.9 |
| Cx. 10-4 | 41.5 | 41.0 | −0.5 | 53.0 | 36.3 | 35.5 | −0.8 | 48.0 |
| Cx. 11-4 | 52.0 | 52.0 | 0.0 | 35.7 | 46.5 | 46.2 | −0.3 | 30.6 |
| Cx. 12-4 | 52.8 | 53.6 | 0.8 | 35.5 | 47.7 | 47.7 | 0.0 | 39.0 |
| Ex. 1-4 | Not Landed (>140) | | | 5° or | 108.1 | 108.6 | 0.5 | 35.2 |
| | | | | less | | | | |
| Ex. 2-4 | Not Landed (>140) | | | 5° or | 124.1 | 121.6 | −2.5 | 24.0 |
| | | | | less | | | | |
| Ex. 3-4 | Not Landed (>140) | | | 5° or | 127.1 | 125.3 | −1.8 | 26.5 |
| | | | | less | | | | |
| Ex. 4-4 | 118.5 | 119.1 | 0.6 | 51.4 | 113.2 | 113.3 | 0.1 | 25.5 |
| Ex. 5-4 | 115.0 | 115.3 | 0.3 | 44.1 | 110.0 | 109.5 | −0.5 | 34.7 |
| Ex. 6-4 | 112.6 | 112.8 | 0.2 | 39.0 | 107.5 | 107.1 | −0.4 | 24.7 |
| Ex. 7-4 | 83.8 | 83.4 | −0.4 | 63.2 | 73.7 | 71.1 | −2.6 | 36.3 |
| Ex. 8-4 | 84.7 | 84.9 | 0.2 | 62.7 | 66.1 | 63.0 | −3.1 | 60.1 |
| Ex. 9-4 | 100.8 | 101.0 | 0.2 | 53.4 | 96.5 | 94.4 | −2.1 | 39.9 |
| Ex. 10-4 | 78.9 | 79.0 | 0.1 | 69.9 | 25.8 | 14.5 | −10.6 | 45.5 |
| Ex. 11-4 | 84.5 | 84.0 | −0.5 | 68.6 | 79.1 | 69.0 | −10.1 | 52.3 |
| Ex. 12-4 | 103.9 | 103.9 | 0.0 | 57.4 | 98.1 | 98.4 | 0.3 | 42.3 |

TABLE 12

| | | Operational | |
|---|---|---|---|
| | | easiness in re- | |
| Liquid Drop Amount | | placing culture | Handling |
| 70 μL | Spheroidization | medium | easiness |
| Cx. 1-4 | X | Δ | Δ |
| Cx. 2-4 | X | Δ | Δ |
| Cx. 3-4 | X | Δ | Δ |
| Cx. 4-4 | X | Δ | Δ |
| Cx. 5-4 | X | Δ | Δ |
| Cx. 6-4 | X | Δ | Δ |
| Cx. 7-4 | X | Δ | Δ |
| Cx. 8-4 | X | Δ | Δ |
| Cx. 9-4 | X | Δ | Δ |
| Cx. 10-4 | X | Δ | ◯ |
| Cx. 11-4 | X | Δ | Δ |
| Cx. 12-4 | X | Δ | Δ |
| Ex. 1-4 | ◯ | ◯ | Δ |
| Ex. 2-4 | ◯ | ⊚ | Δ |
| Ex. 3-4 | ◯ | ⊚ | Δ |
| Ex. 4-4 | ◯ | Δ | Δ |
| Ex. 5-4 | ◯ | ◯ | Δ |
| Ex. 6-4 | ◯ | ◯ | Δ |
| Ex. 7-4 | ◯ | Δ | Δ |
| Ex. 8-4 | ◯ | Δ | ◯ |
| Ex. 9-4 | ◯ | ◯ | Δ |
| Ex. 10-4 | Δ | X | ◯ |
| Ex. 11-4 | Δ | Δ | ◯ |
| Ex. 12-4 | ◯ | ◯ | Δ |

As described above, according to an embodiment of the present invention, a three-dimensional culture method is provided which enables excellent operational easiness or mass productivity and/or which can produce a spheroid with high reproducibility of tissue as compared with conventional three-dimensional culture methods.

Like a synthetic polymer film with a surface which has the moth-eye structure illustrated in Examples, a structure for three-dimensional culture with a solid surface which has a plurality of raised portions whose height is not less than 10 nm and not more than 1 mm is suitably used in a drop culture method. The thus-configured structure for three-dimensional culture is realized by, for example, adhering the above-described synthetic polymer film to the inner bottom surface of a petri dish. That is, the structure for three-dimensional culture can be provided in the form of, for example, a container such as a petri dish or the like.

When the thus-configured structure for three-dimensional culture (for example, container) is used in drop culturing, a structure for three-dimensional culture (for example, container) can be produced which has at the surface a spheroid with higher reproducibility of tissue than conventional ones. The thus-configured structure for three-dimensional culture with a spheroid at the surface is suitably used in drug discovery and research and development in regenerative medicine.

### INDUSTRIAL APPLICABILITY

A three-dimensional cell culture method of an embodiment of the present invention can be widely used in drug discovery, regenerative medicine, etc.

### REFERENCE SIGNS LIST

10S solid surface
10Sp raised portion
12C cell
14M culture medium
16D liquid drop

1. A three-dimensional culture method comprising:
providing a cell suspension, the cell suspension containing a cell and a culture medium;
providing a solid surface, the solid surface having a plurality of raised portions whose height is not less than 10 nm and not more than 1 mm;
attaching a liquid drop of the cell suspension to the solid surface; and
culturing the cell in the liquid drop under such conditions that a direction of gravity exerted on the liquid drop is toward the solid surface.

2. The method of claim 1 wherein, when viewed in a normal direction of the solid surface, a two-dimensional size of the plurality of raised portions is in the range of not less than 10 nm and not more than 500 nm.

3. The method of claim 1, wherein the height of the plurality of raised portions is not less than 10 nm and not more than 500 nm.

4. The method of claim 1, wherein an adjoining distance of the plurality of raised portions is not less than 10 nm and not more than 1000 nm.

5. The method of claim 1, wherein the plurality of raised portions have a generally-conical tip portion.

6. The method of claim 1, wherein a contact angle of the solid surface with respect to the cell suspension is not less than 17°.

7. The method of claim 1, wherein a contact angle of the solid surface with respect to the cell suspension is not less than 90°.

8. The method of claim 1, wherein a sliding angle of the solid surface with respect to the cell suspension is not less than 45°.

9. The method of claim 1, wherein the solid surface is made of a synthetic polymer.

10. The method of claim 1, wherein a volume of the liquid drop is not less than 10 μL and not more than 50 μL.

11. The method of claim 1, wherein a seeding density of the cell contained in the liquid drop is not less than 10³ cells/mL and not more than 10⁷ cells/mL.

12. The method of claim 1, wherein a height of the liquid drop is not less than 1 mm.

13. The method of claim 1, further comprising adding the culture medium into the liquid drop while the cell is cultured in the liquid drop.

14. The method of claim 13 further comprising, before adding the culture medium, aspirating part of the culture medium from the liquid drop.

15. A structure for three-dimensional culture having a solid surface for use in the three-dimensional culture method as set forth in claim 1.

16. A method for producing a structure for three-dimensional culture, the structure for three-dimensional culture having the solid surface, the structure for three-dimensional culture including on the solid surface a spheroid cultured using the three-dimensional culture method as set forth in claim 1.
